(19) United States
(12) Patent Application Publication
(10) Pub. No.: US 2008/0228273 A1
(43) Pub. Date: Sep. 18, 2008

(54) Implants

(76) Inventors: Alan Rory Mor McLeod, Somerset (GB); Christopher Reach, Taunton (GB); Raymond Ross, Manchester (GB); Andre Jackowski, Birmingham (GB)

Correspondence Address: JONATHAN SPANGLER;NU VASIVE, INC., 4545 TOWNE CENTRE COURT, SAN DIEGO, CA, 92121, US

(21) Appl. No.: 11/994,410

(22) PCT Filed: Jul. 20, 2006
(86) PCT No.: PCT/US06/28411

§ 371 Date: Dec. 31, 2007

(30) Foreign Application Priority Data
Jul. 20, 2005 (GB) 0514891.1

(51) Int. Cl. A61F 2/44

(52) U.S. Cl. 623/17.11

(57) ABSTRACT

Disc prostheses, particularly for use in the lumbar region of the spine are provided. The disc prosthesis includes an outer component manufactured at least in part from fibre and at least one filling element introduced into the outer component in situ.

### CROSS-REFERENCES TO RELATED APPLICATIONS

This application is an International Patent Application claiming the benefit of priority from commonly-owned and co-pending British Patent Application No. 0514891.1, entitled “Improvements in and Relating to Implants,” and filed on Jul. 20, 2005 the entire contents of which are expressly incorporated into this disclosure by reference as if set forth in their entirety herein.

### BACKGROUND

1. Field of the Invention

This invention concerns improvements in and relating to implants and more particularly (but not exclusively) to spinal implants for full or partial replacement of the nucleus pulposus.

2. Related Art

Intervertebral discs within a spine perform a variety of functions. Discs provide proper spacing between vertebral bodies and allow the intervertebral foramen to maintain its height, allowing the segmental nerve roots room to exit each spinal level without compression. Discs also provide shock absorption, which not only allows the spine to compress and rebound when the spine is axially loaded (during such activities as jumping and running) but also to resist the downward pull of gravity on the head and trunk during prolonged sitting and standing. Discs also allows motion coupling based on their elasticity, so that the segment may flex, rotate, and laterally bend all at the same time during a particular activity. This would be impossible if each spinal segment were locked into a single axis of motion.

An intervertebral disc consists of four distinct parts: the nucleus pulposus, the annulus fibrosus, and the end plates of the adjacent vertebrae. It should be noted that although these four sections are very much distinct in their own right the boundaries between then are not as distinct. Most investigators tend to ignore the end plates and dismiss them as merely as the barrier between the vertebrae and the parts of the disc which allow motion of the spine. However, the end plates are important in completing the structure of the disc and creating some of the boundary conditions that define the behavior of the disc.

The annulus is the outer ring of the disc. The annulus comprises a strong, laminated structure of opposed layers of Collagen fibres. An annulus typically comprises around 12 laminae, with 6 provided in each direction of fibre travel. The layers are at an angle of approximately 30° on every other layer, with 30° in the opposite direction on the remaining layers. The functions the annulus performs determine the need for this type of structure. No matter which direction the vertebrae moves, there will always be some fibres in tension and some in compression. Thus, the annulus will always be acting using some fibres to stretch (they will resist stretch like an elastic band) and pull the spine back into the correct posture. The annulus has overlapping, radial bands, not unlike the plies of a radial tire, and this allows torsional stresses to he distributed through the annulus under normal loading, without rupture. One study suggested that the posterior part of the annulus is the weaker side, so more susceptible to damage—Tsuji; Structural variation of the annulus fibrosis. Spine 18 pp 204-²10, 1993. The annulus is the strongest part of the disc.

The nucleus at the centre of the disc is a highly hydrated gel of proteoglycans. In children and young adults, the water content can account for up to 80% of its weight—Ghosh. This gel material is a very thick fluid that is dense enough to be able to be torn. It serves the twin purposes of both direct load bearing and, by being fluid in nature, being able to change shape under loading to distribute the load to the annulus. The nucleus may only bear half the load of the FSU (functional spinal unit) with the annulus carrying the rest—Finneson; Low back pain. ISBN 0-397-50493-4, 1992. It is this shared loading that allows the disc to continue to operate even after the nucleus has been damaged.

The end plates are composed of hyaline cartilage. This is basically a “hydrated Proteoglycan gel, reinforced by Collagen Fibrils”—Ghosh; The Biology of the intervertebral Disc. CRC Press, ISBN 084936711523. As stated, the boundary between the annulus and end Plate is not a distinct one, under a microscope the two parts merge together, with a region which is neither one tissue nor the other.

Degeneration and/or herniation and/or damage to a disc can occur during a patient's life. From around the 20th year of a person's life, the discs become completely avascular, although they show high metabolic turnover. The water content of the discs will decrease the older the person gets. Degenerative disc disease (DDD) is the process of a disc losing some of its function, due to a degenerative process, and is a very common and natural occurrence. At birth the disc is comprised of about 80% water. As ageing occurs, the water content decreases and the disc becomes less of a shock absorber, the proteins within the disc space also alter their composition.

The relationship between degeneration and pain is not a clear one. One theory to explain why some degenerative discs are painful is that a tear in the annulus may release nucleus material, which is known to be inflammatory. Another theory is that nerve ingrowth occurs into the discs, in that some people seem to have nerve endings that penetrate more deeply into the outer annulus than others, and this is thought to make the disc susceptible to becoming a pain generator. A still further theory is that a loss of disc height occurs as the water content lowers, which may cause the disc to bulge outwards and pressurizes the nerve roots (thus causing pain). A loss in height may have other pain generating effects, including an alteration of the biomechanics, wherein the nucleus will loses its ability to pressurize the annulus and the annulus itself will be forced to carry the compressive load at that level in the spine. A loss is disc height may also affect the load distribution, wherein the load will not be carried in an even manner throughout the disc. Alterations in disc biomechanics may also affect both the patient's range of motion and alter the position of the instantaneous axis of rotation in normal movements. The result of these factors will usually mean increased loading on the facet joints that may in turn start to degenerate and become symptomatic. What ever the reason behind the degeneration causing the pain, treatment to improve the position and the patient's life is important. The treatment options are discussed in more detail below.

A herniated disc is similar to a prolapsed one, in that there is a bulge in the disc itself however, the disc will not have collapsed in the same way. The injury is thought to be though a combination of a degenerative process and mechanical loading. The stages of disc herniation Ibrahim; ‘Colorado spine institute; http://www.coloradospineinstitute.com 2004; are disc degeneration, perhaps due to chemical changes associated with aging cause the disc to weaken; formation of a bulge due to this localized failure of the annulus; progression of the condition can cause the nucleus to protrude out as a herniation; the bulge will press against the nerves in the spinal canal and cause pain that the body sees as coming from the legs; further progression results in extrusion as the gel-like nucleus pulposus breaks through the annulus fibrosis, but remains within the disc; further progression may result in the nucleus pulposus breaking though time annulus fibrosus and lying outside the disc in the spinal canal, a sequestered disc.

When a disc that is showing signs of degeneration or herniation or disease or damage become painful a surgeon may often operate. Treatments that may be conducted include partial discectomy (removal of local annular material to the site of a herniation), partial nucleotomy (removal of local nucleus material close to the site of the herniation), and discectomy and fusion (removal of the entire disc and fusion of the disc space, used in more serious cases). Other treatments include disc replacement and nucleus replacement, which are both relatively new treatments used as an alternative to fusion.

### SUMMARY OF THE INVENTION

According to a first aspect of the invention we provide an implant, the implant including an outer component and one or more filling elements provided within the outer component. Preferably the implant is a spinal implant. The implant may be a partial nucleus pulposus replacement. The implant may be a total nucleus replacement. Preferably the implant maintains the separation of the vertebrae which it is provided between. The implant may mimic the characteristics of a naturally occurring nucleus. Preferably the implant provides some or all of the resistance to compressive loads provided by a natural nucleus. The implant may be inserted anteriorly or posteriorly. The implant may be provided within a natural annulus and/or an artificial annulus. The implant may include a radio-opaque element, for instance a marker. The present invention is intended to be particularly useful as part one or more of the following treatments: Nucleotomy to replace the lost or removed nucleus material by inserting the implant to provide a nucleus material equivalent alongside the remaining nucleus material; Artificial Disc Replacement to restore a functional nucleus by inserting the implant in conjunction with an annulus replacement; and Artificial Nucleus Replacement to restore a functional nucleus by inserting the implant.

The outer component may be porous. The outer component may be a bag or other form of container. The outer component may have an opening to permit the insertion of the one or more filling elements. Preferably the opening is closable, for instance by one or more of folding, stitching, suturing, gluing, stapling or the like. The opening may be closed by a flap. A plurality of flaps may be provided. One or more flaps may extend across at least a part of the opening from one side, potentially with one or more flaps extending across at least a part of the opening from the other side. The flaps from one side may alternate with one or more flaps from the other side.

The outer component may be made of fabric, particularly a woven fabric, and may be of textile. The outer component may be made of fibres, for instance mono-filaments, multi-filaments, braided filaments, twisted filaments, twisted multi-filaments or mixtures thereof. The fabric may be one or more of flat woven, circular woven, 3-D woven, knitted, braided, embroidered or combinations thereof. The fabric may include and/or be formed from one or more fibre materials. The fabric may include and/or be formed of one or more of polyester, polypropylene, polyethylene, ultra high molecular weight polyethylene, glass fibre, glass, polyararnide, metal, copolymers, polylactic acid, polyglycolic acid, biodegradable materials, silk, cellulose or polycaprolactone.

The outer component may have one or more pores. Preferably the pores in the outer component have at least one cross-sectional dimension that is less than the smallest cross-sectional dimension of the filling elements. Preferably the cross-sectional area of the pores is less than the minimum cross-sectional area of the filling elements. Preferably the filling elements cannot pass through the pores of the outer component, particularly from the inside to the outside and/or pass through without external intervention. The filling elements may pass though the pores when subjected to external intervention, particularly from the outside to the inside.

The outer component may entirely surround the filling elements and/or encapsulate the filling elements. One or more apertures or gaps may be provided in the outer component, ideally to provide or assist fluid communication through the outer component. Preferably a large number of apertures or gaps are provided in the material from which the outer component is formed, for instance a woven fabric. The apertures or gaps occurring in the outer component may be due to the manner of manufacture of the material from which it is formed or may be supplemented with further apertures or gaps. For instance the pores may be defined between the fibres of the fabric or textile. For instance, the pores may be made by making holes. The supplementation may be provided by degradation and/or absorption of one or more materials forming the outer component. Where apertures or gaps are provided, preferably they have at least one cross-sectional dimension that is less than the smallest cross-sectional dimension of the filling elements. Preferably the cross-sectional area of the gaps or apertures is less than the minimum cross-sectional area of the filling elements. Preferably the filling elements cannot pass through the gaps or apertures of the outer component.

The outer component may constrain the one or more filling elements. The outer component may have a first profile, preferably when empty of filling material. The outer component may have a second profile, preferably when full or partially full of filling material. Preferably the second profile for the outer component is configured to match the void in the disc space to be filled. The second profile may be pre-configured, for instance by selecting a shape and/or size of outer component. The selection may be based upon a measurement of the void in the disc space, for instance using an inflatable device, such as a balloon, potentially made of or including radio-opaque material. The second profile may be post-configured or configured after implantation, for instance by expanding the outer component to assume the profile of the void in the disc space. A combination of pre and post-configuration may be used. The second profile may be spherical. The second profile may be a disc shape, for instance with a top and bottom surface perpendicular to one or more side surfaces. The top and bottom surfaces may be parallel to one another or inclined relative to one another.

The outer component may be configured and/or formed of and/or provided with one or more materials intended to promote tissue growth, particularly tissue ingrowth through the outer component and/or between the outer component and one or more of the filling elements and/or between two or more of the filling elements and/or into the material of the outer component and/or into the material of the one or more filling elements. Tissue growth between the outer component and/or an element thereof and the filling elements may be provided. Tissue growth between elements of the outer component may be provided, for instance between and/or though elements projecting from the outer component, for instance loops. Tissue growth may be promoted by the material type, for instance polyester. Tissue growth may be promoted by the configuration, particularly the size and/or number of pores and/or gaps and/or apertures in the outer component. Tissue growth may be promoted by a chemical, for instance a pharmaceutical, provided as part of the outer component or associated therewith.

Tissue ingrowth may provide for the replacement of disc material removed during the surgery, for instance diseased material and/or material removed to make room for the implant and/or removed to provide access to another location, for instance to a pathology. Tissue ingrowth may provide for the restoration of disc height and/or nucleus function and/or annulus function and/or intervertebral separation lost due to disease and/or degeneration and/or surgery.

The outer component may be configured and/or formed of and/or provided with one or more materials intended to inhibit or prevent tissue growth, particularly tissue ingrowth through the outer component and/or between the outer component and one or more of the filling elements and/or between two or more of the filling elements and/or into the material of the outer component and/or into the material of the one or more filling elements. Tissue ingrowth may be inhibited or prevented by the material type, for instance polyethylene. Tissue ingrowth may be inhibited or prevented by the configuration, particularly the size and/or number of pores and/or gaps and/or apertures in the outer component. Tissue ingrowth may be inhibited or prevented by a chemical, for instance a pharmaceutical, provided as part of the outer component or associated therewith. The outer component may he configured and/or formed of and/or provided with one or more materials to inhibit or prevent tissue growth in one or more areas and/or one or more directions and to promote or allow tissue growth in one or more other areas and/or one or more other directions. For instance, tissue ingrowth may be promoted in the region of and/or towards the outer and/or adjacent end plates, and/or with tissue ingrowth being inhibited in the region of and/or towards the anterior/posterior plane and/or towards the esophagus and/or major vessels and/or spinal canal.

One or more materials used in the outer component may be bio-absorbable. The bio-absorbable material may be used to decrease the amount of outer component present and/or positions at which the outer component is present and/or density at which the outer component is present overtime. The bio-absorbable material may restrain the outer component in a first state, the bio-absorption of the material allowing the outer component to assume a second state. The second state may provide a greater internal volume for the outer component and/or greater porosity for the outer component and/or reduction in mass of the outer component and/or provide more space for tissue ingrowth.

Bio-absorbable material may be incorporate in the outer component by providing areas of bio-absorbable material and/or one or more fibres of bio-absorbable material. The outer component may be entirely bio-absorbable or only partially. Different materials having different rates of bio-absorption may be used for different areas and/or different fibres within the outer component. Slow, moderate and fast bio-absorption materials may be used.

The outer component may be provided with an external surface, ideally modified, at least in part, to increase adhesion between the outer component and the tissue and/or bone it is in contact with. The outer component may be provided with an external surface, ideally modified, at least in part, to increase load transfer between the outer component and the tissue and/or bone it is in contact with. The outer component may be provided with an external surface, ideally modified, at least in part, to resist movement between the outer component and the tissue and/or bone it is in contact with. The tissue and/or bone it is in contact with may be the tissue and/or bone present on implantation and/or the tissue and/or bone it comes into contact with after implantation, for instance due to ingrowth. The outer component may be provided with an external surface which is at least in part provided with a rough surface and/or surface with protrusions. The roughness and/or protrusions may be provided by variations in the form and/or configuration and/or properties of the outer component. For instance, the tensions of fibres forming the outer component may vary between fibres and/or between different parts of the same fibre. The outer component may be provided with one or more loops and/or projections.

The outer component may be provided with an internal surface, ideally modified, at least in part, to increase adhesion between the outer component and the one or more inner components and/or one or more filling elements and/or the tissue or ingrowth material, preferably associated with the one or more filling elements, it is in contact with. The outer component may be provided with an internal surface, ideally modified, at least in part, to increase load transfer between the outer component and the one or more inner components and/or one or more filling elements and/or the tissue or ingrowth material, preferably associated with the one or more filling elements, it is in contact with. The outer component may be provided with an external surface, ideally modified, at least in part, to resist movement between the outer component and the one or more inner components and/or one or more filling elements and/or the tissue or ingrowth material, preferably associated with the one or more filling elements, it is in contact with. The tissue and/or bone it is in contact with may be the tissue and/or bone present on implantation and/or the tissue and/or bone it comes into contact with after implantation, for instance due to ingrowth. The outer component may be provided with an internal surface which is at least in part provided with a rough surface and/or surface with protrusions. The roughness and/or protrusions may be provided by variations in the form and/or configuration and/or properties of the outer component. For instance, the tensions of fibres forming the outer component may vary between fibres and/or between different parts of the same fibre. The outer component may be provided with one or more loops and/or projections extending towards the one or more inner components and/or one or more filling elements.

The implant may include a further element. A plurality of further elements may be provided. The further element may be a structural element. The further element may be a resilient element. The further element may be a stiffening element. The further element may be linear or include one or more linear parts. The further element may be curved or include one or more curved parts. The further element may be or include a ring and/or coil and/or loop and/or wave and/or spiral and/or zig-zag. The further element may be or include fibres, for instance mono-filaments, multi-filaments, braided filaments, twisted filaments, twisted multi-filaments. The further element may be or include metal. The further element may be or include plastics. The further element may be or include a shape memory material. The further element may be a wire. The further element may be a fibre.

In one preferred embodiment, the further element may be provided as a part of the outer component and/or one or more of the inner components, ideally an integral part thereof. The further element may be a fibre or part of a fibre of the outer component and/or one or more inner components. The further element may be a fibre or part of a fibre which is provided from a different material and/or thickness and/or cross-sectional area to another fibre or part of the fibre in the outer component.

In another preferred form, the further element may be provided separate from the outer component and/or one or more inner components. The further element may be in contact with the outer component and/or one or more inner components. The further element may be inside and/or outside the outer component and/or one or more inner components. The further element may pass through the outer component and/or one or more inner components on one or more occasions. The further element may be woven into the outer component and/or one or more inner components or may have the outer component and/or one or more inner components woven around it. The further element may define, at least in part, the profile of the implant and/or outer component and/or one or more of the inner components. The further element may extend around the periphery of the outer component and/or one or more inner components. The further element may, at least in part, define one or more side walls thereof and/or upper and/or lower walls thereof.

The further element may have a rest state and a deformed state. Preferably a force is applied to change the further element from the rest state to the deformed state. Preferably the removal or reduction of the force causes the further element to tend towards the rest state from the deformed state. The force may be a compressive force. Preferably the implant has a smaller cross-sectional profile when viewed in at least one direction when the further element is in the deformed state compared with when it is in the rest state. Preferably the implant is inserted into the disc space in the direction of this view or in a direction 180° thereto. The further element may resist expansion of the outer component and/or one or more inner components beyond a state, for instance the rest state. Thus the further element may resist over expansion. A further element may be provided for a part of the implant with a specific function, for instance near the opening in the outer component through which the one or more filling elements are introduced. The further element may assist the closure of the opening.

The implant, particularly the outer component thereof, may be anchored within the disc space using tissue ingrowth to link it to the tissue and/or bone of its surroundings and/or using one or more sutures and/or one or more staples and/or one or more barbs and/or one or more separate mechanical anchors to link it to the tissue and/or bone of its surroundings. Alternatively or additionally, the implant and particularly its outer component may be anchored by means of one or more parts of the outer component. In particular the one or more parts may be anchors provided on the fabric or textile of the outer component. The anchors may be integral with the outer component, for instance in the form of barbs provided on the fibres of the outer component. The anchors may be bioabsorbable.

One or more further components may be provided, for instance as inner component provided within the outer component. The one or more inner components may provide one or more layers between the outer component and the one or more filling elements. The one or more inner components may be of fabric. The fabric may be formed by flat or circular weaving, knitting, braiding, embroidery or combinations thereof. The fabric may be formed using one or more of polyester, polypropylene, polyethylene, ultra high molecular weight polyethylene, carbon fibre, glass fibre, glass, polyaramide, metal, copolymers, polylactic acid, polyglycolic acid, biodegradable materials, silk, cellulose, silk worm silk, spider silk or polycaprolactone.

Preferably the one or more inner components are separate from the one or more filling elements. Preferably the one or more inner components are separate from the outer component. Relative movement may be facilitated between the inner and outer components. Relative movement between the inner component(s) and one or more filling elements may be allowed. Preferably movement between the inner and outer components is greater than between the inner component and one or more filling elements. Preferably movement between the inner and outer components is facilitated in preference to movement between the inner component and one or more filling elements. Preferably any movement, particularly sliding movement, within the implant is greater between the outer component and inner component than between the inner component and one or more filling elements.

The one or more inner components may entirely surround the one or more filling elements and/or encapsulate the one or more filling elements. One or more apertures or gaps are preferred in the one or more inner components, ideally to provide fluid communication though the one or more inner components. Preferably a large number of apertures or gaps are provided in the material from which the one or more inner components is formed, for instance utilizing a woven fabric. The apertures or gaps may occur due to the manner of manufacture of the material from which it is formed may be supplemented with further apertures or gaps. The supplementation may be provided by degradation and/or absorption of one or more materials forming the one or more inner components.

The one or more inner components may be configured and/or formed of one or more materials intended to promote tissue growth, particularly tissue ingrowth between the one or more inner components and the core and/or through the one or more inner components. One or more materials used in the one or more inner components may be bioabsorbable and/or soluble and/or degradable, particularly within the spine. The bioabsorbable material may be used to decrease the amount of one or more inner components present and/or positions at which the one or more inner components are present and/or density at which the one or more inner components are present overtime. Areas of bioabsorbable material may be provided. Bio-absorbable fibres may be used to form the one or more inner components. The one or more inner components may be entirely bioabsorbable or only partially. Different materials having different rates of bio-absorption may be used. They may be mixed together in the one or more inner components and/or may be used for particular areas thereof and/or in a particular sequence within the one or more inner components. Slow, moderate and fast bio-absorption materials may be used. Preferably bio-absorption of the one or more inner components is used to provide space for tissue ingrowth.

The inner component may provide a smooth inner surface which potentially contacts the one or more filling elements. Preferably uniform contact between the inner surface of the inner component and the one or more filling elements is provided. Preferably the fibres forming the inner surface of the inner component are evenly positioned with respect to one another. Preferably any abrasion of the one or more filling elements by the inner component is distributed rather than localized. The inner component preferably provides a smooth inner fabric surface, and ideally woven fibrous surface. A densely packed material may be used for the inner surface, ideally to provide the uniform contact surface with the one or more filling elements. The inner surface of the inner component may be of a different material and/or different configuration to the inside and/or outer surface of the inner component.

The one or more inner components may have fewer and/or smaller apertures, gaps or pores than the outer component. The one or more inner components may be formed with a tighter weave than the outer component. The inner component may be provided with an external surface, ideally modified, at least in part, to increase adhesion between the inner component and the outer component and/or the tissue and/or bone it is in contact with. The inner component may be provided with an external surface, ideally modified, at least in part, to increase load transfer between the inner component and the outer component and/or the tissue and/or bone it is in contact with. The inner component may be provided with an external surface, ideally modified, at least in part, to resist movement between the inner component and the outer component and/or the tissue and/or bone it is in contact with. The tissue and/or bone it is in contact with may he the tissue and/or bone present on implantation and/or the tissue and/or bone it comes into contact with after implantation, for instance due to ingrowth. The inner component may be provided with an external surface which is at least in part provided with a rough surface and/or surface with protrusions. The roughness and/or protrusions may be provided by variations in the form and/or configuration and/or properties of the inner component. For instance, the tensions of fibres forming the inner component may vary between fibres and/or between different parts of the same fibre. The inner component maybe provided with one or more loops and/or projections.

The inner component may be provided with an internal surface, ideally modified, at least in part, to increase adhesion between the inner component and one or more filling elements and/or the tissue or ingrowth material, preferably associated with the one or more filling elements, it is in contact with. The inner component may be provided with an internal surface, ideally modified, at least in part, to increase load transfer between the inner component and the one or more filling elements and/or the tissue or ingrowth material, preferably associated with the one or more filling elements, it is in contact with. The inner component may be provided with an internal surface, ideally modified, at least in part, to resist movement between the inner component and the one or more filling elements and/or the tissue or ingrowth material, preferably associated with the one or more filling elements, it is in contact with. The tissue and/or bone it is in contact with may be the tissue and/or bone present on implantation and/or the tissue and/or bone it comes into contact with after implantation, for instance due to ingrowth. The inner component may be provided with an internal surface which is at least in part provided with a rough surface and/or surface with protrusions. The roughness and/or protrusions may be provided by variations in the form and/or configuration and/or properties of the inner component. For instance, the tensions of fibres forming the inner component may vary between fibres and/or between different parts of the same fibre. The inner component may be provided with one or more loops and/or projections extending towards the one or more filling elements.

The one or more filling elements may be fibrous and/or formed of single filaments. The one or more filling elements may be a single component. The filling elements may be formed of multiple components. A single or multiple components may be provided within an inner component, such as a jacket. Where multiple components are used it is possible that each is provided within its own inner component. The component may be provided within its own inner component in-situ or before insertion into the disc space. The filling elements may be blocks, beads, spheres, cylinders, rods or other such elements. The one or more filling elements may be formed by folded and/or bending and/or spiraling one or more elements within the outer component.

One or more of the one or more filling elements may include and/or be formed from one or more fibre materials. One or more of the one or more filling elements may include and/or be formed of one or more of polyester, polypropylene, polyethylene, glass fibre, glass, polyaramide, metal, copolymers, polylactic acid, polyglycolic acid, biodegradable materials, silk, cellulose or polycaprolactone.

One or more of the filling materials may include or be biological material, for instance material collected from an animal or human. The material may be from a spine, for instance a disc and preferably the nucleus thereof. The material may be collected from the patient. The material may be collected from a spinal disc of the patient. The disc may be the disc receiving the implant or another disc. The material may be collected in making the space to receive the implant. The material may be a mixture.

One or more of the filling elements may include and/or be formed from an elastomeric material, visco-elastic material, silicone based materials and/or polyurethane based materials and/or silicone/polyurethane blends and/or copolymers and/or mixes and/or hydrogel. One or more of the filling elements may be provided in a form which can flow into the space defined by the outer component, preferably with the filling elements then setting. The one or more filling elements may be impregnated and/or doped and/or provided with further materials. The further materials may be or include barium sulphate.

Preferably one or more filling elements that are porous and/or define voids within themselves and/or between parts of a filling element are provided. The pores and/or voids and/or apertures and/or gaps provided in or by the filling elements ideally provide fluid communication through the filling elements and/or there between. Preferably a large number of pores and/or voids and/or apertures and/or gaps are provided in the material from which filling elements are formed. Preferably a large number of pores and/or voids and/or apertures and/or gaps are provided by one or more of the filling elements. Preferably a large number of pores and/or voids and/or apertures and/or gaps are provided within one or more of the filling elements by virtue of their structure.

One or more filling elements may be formed of unconstrained fibres. One or more filling elements may be formed of unbraided fibres. One or more filling elements may be formed of braided fibres. One or more filling elements may be formed of twisted fibres. One or more filling elements of felt or felt-like material may be provided. One or more filling elements with interlaced fibres may be provided. One or more filling elements may be provided with aligned fibres. One or more filling elements may be provided with one or more groups of aligned fibres and/or one or more non-aligned fibres and/or one or more groups of fibres on different alignments to the first. One or more filling elements with non-linear fibres may be provided. One or more filling elements with wavy and/or curved and/or zig-zag fibres may be provided. One or more filling elements with fibres which act to space each other from one another may be provided. One or more filling elements with primary fibres having a first alignment and secondary fibres on a different alignment, which serve to space the primary fibres from one another may be provided. One or more filling elements of cotton wool or like material may be provided.

One or more filling elements with fibres of two or more different cross sections maybe provided. The fibres of different cross sections may be linear and/or non-linear. One or more filling elements with fibres provided in a first direction may be provided, with one or more restraining fibres or material. The restraining fibres and/or material may surround and/or enclose and/or be wrapped around and/or contact a plurality of fibres. The restraining fibre or material may be provided as a band. The restraining fibres of material may be provided at the ends of the filling elements and/or at intermediate locations thereon. One or more filling elements may be provided with peripheral fibres or material provided around the filling element. The peripheral fibres or material may be wrapped around the filling elements in a spiral manner and/or criss-cross manner. The fibres or material may be provided in an anti-clockwise and/or clockwise manner. A fishnet of fibres may be provided around one or more filling elements.

One or more filling elements may be provided with pieces provided therein. The pieces may be intermixed with one or more fibres. The pieces may be spheres, beads, blocks or the like. The pieces may be integral with the fibres and/or connected thereto and/or free to move relative to the fibres. Preferably fibres are wrapped and/or extend around at least part of the periphery of the beads, ideally in a variety of directions. The pieces may be linked together by a fibre or filament, particularly in the case of the series of spheres. The spheres may be surrounded by a mass of braided fibres. The masses of braided fibres may be linked by one or more fibres or filaments. Preferably the masses of fibres surround the spheres.

A single layer of filling elements may be provided within the outer component. Multiple layers of filling elements may be provided within the outer component. One or more intermingled filling elements may be provided within the outer component. The filling elements may be of linear configuration and/or curved and/or wavy. One or more spiral filling elements may be provided. One or more filling elements of substantially circular cross-section may be provided. One or more filling elements with one or more flat surfaces may be provided. One or more filling elements of generally square and/or pentagonal and/or hexagonal and/or octagonal cross-section may be provided.

The pores and/or voids and/or apertures and/or gaps occurring in the filling elements and/or there between may be due to the manner of manufacture of the material from which it is formed or may be supplemented with further pores and/or voids and/or apertures or gaps. The supplementation may he provided by degradation and/or absorption of one or more materials forming the filling elements.

The one or more filling elements may be configured and/or formed of one or more materials intended to promote tissue growth, particularly tissue ingrowth through one or more filling elements and/or between the outer component and one or more filling elements and/or between two or more of filling elements and/or into the material of the outer component and/or into the material of the one or more filling elements. Tissue growth may be promoted by the material type, for instance polyester, included in one or more filling elements. Tissue growth may be promoted by the configuration, particularly the size and/or number of pores and/or gaps and/or apertures in one or more filling elements.

Tissue ingrowth may provide for the replacement of disc material removed during the surgery, for instance diseased material and/or material removed to make room for the implant and/or removed to provide access to another location, for instance to a pathology. Tissue ingrowth may provide for the restoration of disc height and/or nucleus function and/or annulus function and/or intervertebral separation lost due to disease and/or degeneration and/or surgery.

The one or more filling elements may be configured and/or formed of one or more materials intended to inhibit or prevent tissue growth, particularly tissue ingrowth through one or more filling elements and/or between the outer component and one or more filling elements and/or between two or more of filling elements and/or into the material of the outer component and/or into the material of the one or more filling elements. Tissue ingrowth may be inhibited or prevented by the material type, for instance polyester, included in one or more filling elements. Tissue ingrowth may be inhibited or prevented by the configuration, particularly the size and/or number of pores and/or gaps and/or apertures in one or more filling elements.

One or more materials used in one or more of the filling elements may be bioabsorbable. The bio-absorbable material may be used to decrease the amount of one or more filling elements present and/or positions at which one or more filling elements is present and/or density at which one or more filling elements is present overtime. The bioabsorbable material may restrain one or more of the filling elements, or a part thereof in a first state, the bio-absorption of the material allowing one or more filling elements, or a part thereof, to assume a second state. The second state may provide a greater internal volume for one or more filling elements and/or greater porosity for one or more filling elements and/or reduction in mass of one or more filling elements and/or provide more space for tissue ingrowth.

Bio-absorbable material may be incorporate in one of more filling elements by providing areas of bio-absorbable material and/or some fibres of bio-absorbable material. One or more of the one or more filling elements may be entirely bio-absorbable or only partially. Different materials having different rates of bio-absorption may be used for different areas and/or different fibres within one or more filling elements. Slow, moderate and fast bio-absorption materials may be used.

The one or more filling materials may be provided with an external surface, ideally modified, at least in part, to increase adhesion between the one or more filling materials and the outer component and/or one or more inner components and/or the tissue and/or bone it is in contact with. The one or more filling materials may be provided with an external surface, ideally modified, at least in part, to increase load transfer between the one or more filling materials and the outer component and/or the one or more inner components and/or the tissue and/or bone it is in contact with. The one or more filling materials may be provided with an external surface, ideally modified, at least in part, to resist movement between the one or more filling materials and the outer component and/or the one or more inner components and/or the tissue and/or bone it is in contact with. The tissue and/or bone it is in contact with may be the tissue and/or bone present on implantation and/or the tissue and/or bone it comes into contact with after implantation, for instance due to ingrowth. The one or more filling materials may be provided with an external surface which is at least in part provided with a rough surface and/or surface with protrusions. The roughness and/or protrusions may be provided by variations in the form and/or configuration and/or properties of the one or more filling materials. For instance, the tensions of fibres forming the one or more filling materials may vary between fibres and/or between different parts of the same fibre. The one or more filling materials may be provided with one or more loops and/or projections.

Preferably the one or more filling elements and/or outer component and/or inner component and/or combination of two or all thee thereof and/or tissue ingrowth and/or the combination of tissue ingrowth with one or two or thee of the filling elements, outer component and inner component may provide equivalent properties and/or behavior to the nucleus pulposus of a natural disc, for instance during compression and/or distraction and/or horizontal gliding and/or axial rotation and/or fixation and/or extension. The one or more filling elements and outer component in such an embodiment may be formed of different materials and/or formed in different ways and/or be provided with different properties. In particular the one or more filling elements may mimic the properties of the nucleus and the outer component may mimic the properties of the annulus, or properties intermediate the nucleus and annulus.

A single void in a disc space may be provided with a plurality of implants. The implants may be the same as one another or may be different from one another. The implants may be anchored to the disc and/or surrounding tissue and/or surrounding bone. The implants are preferably not connected to one another. One or more of the plurality of implants may be spaced apart from another within the disc space. Two or more of the plurality of implants may be in contact with one another within the disc space.

The first aspect of the invention may include any of the features, options or possibilities set out elsewhere in this document.

According to a second aspect of the invention we provided a surgical technique in which, at least part of a spinal disc is removed and an implant is provided, the implant having an outer component and one or more filling elements provided within the outer component. A part or the whole of a nucleus pulposus may be replaced. The implant may be inserted anteriorly, posteriorly, laterally and/or any combination or variation thereof.

Preferably the outer component is inserted through the same incision as is used to remove the nucleus material. Preferably the incision is only as large as needed for the nucleus material removal stage. The outer component may be folded and/or compressed for insertion into the intervertebral disc space. Preferably the outer component is empty when introduced to the disc space. Preferably the one or more filling elements are absent from the outer component during insertion into the intervertebral disc space.

The outer component and/or implant may be positioned proximal to the incision through which it is inserted. The outer component may be filled at this position, and preferably expands into the disc space away from the incision. The outer component or implant may be positioned distal to the incision though which it is inserted. The inner component may be filled at this position, and preferably expands into the disc space towards the incision. The outer component and/or implant may be positioned in the centre of the nucleus and/or centre of the space formed in the nucleus. The outer component may be filled at this position, and preferably expands into the disc space both towards and away from the incision though which it was inserted.

In one embodiment, the outer component may form an enclosed space without the one or more filling elements present. The one or more filling elements may be introduced into the enclosed space after the outer component has been provided in the disc space. The one or more filling components may be introduced into the enclosed space through one or more apertures, gaps or pores in the outer component, for instance an aperture, gap or pore between fibres of the outer component. The size of the aperture, gap or pore may have a first size and a second size, the second size being larger than the first size. Preferably the one or more filling elements are introduced into the enclosed space with the aperture, gap or pore in the second size. Preferably the aperture, gap or pore is of the first size prior to the one or more filling materials being introduced and/or after the one or more filling materials have been introduced, The aperture, gap or pore may change from the first size to second size due to the introduction of a filling element insertion tool or the filling element itself. The aperture, gap or pore may change from the second size to the first size due to the removal of a filling element insertion tool or the passage of a filling element into the enclosed space. The filling element may be unable to pass through the aperture, gap or pore with the aperture, gap or pore in the first size. Preferably the filling element is able to pass through the aperture, gap or pore with the aperture, gap or pore in the second size. The one or more filling elements may be introduced by injection.

The outer component may be inserted into the disc space with one or more filling elements inside the outer component. All of the one or more filling elements may be present in the outer component prior to insertion into the disc space. Preferably the one or more filling elements are introduced into the outer component within the intervertebral disc space. The one or more filling elements may be deployed from an applicator, for instance by extrusion therefrom. Preferably the one or more filling elements are provided through the incision used to remove the nucleus material. Preferably the incision used for introducing the one or more filling elements is no larger than the incision necessary for the removal of the nucleus material.

Preferably the technique includes a first time in which the implant provides one or more characteristics of a naturally occurring disc by virtue of a non-biological mechanism, and a second time at which the implant provides one or more characteristics of naturally occurring disc by a combination of a non-biological mechanism and biological mechanism. Ideally, the biological mechanism is tissue in-growth. The technique may include a third time with substantially all of the one or more characteristics of a naturally occurring disc are provided by a biological mechanism. Preferably the transition from the mechanism at the first time to the second time and/or third time is due to bio-absorption of one or more of the materials forming the implant and particularly forming one or more filling elements thereon.

The second aspect of the invention may include any of the features, options or possibilities set out elsewhere in this document, including in the first aspect.

### BRIEF DESCRIPTION OF THE DRAWINGS

Various embodiments of the invention will now be described, by way of example only, and with reference to the accompanying drawings in which:

FIG. 1 shows a perspective view of a disc featuring part of a device according to an embodiment of the present invention;

FIG. 2 shows the view of FIG. 1 with the device near completion;

FIG. 3 shows the dispensing of one embodiment of the filling using one embodiment of an applicator;

FIGS. 4a to 4c show other embodiments of fillings;

FIG. 5 shows a further embodiment of a filling in perspective view;

FIGS. 6a and 6b shows still further embodiments of filings in perspective view;

FIG. 7 shows yet another embodiment of a filling;

FIG. 8 shows an embodiment of the invention including beads;

FIG. 9 shows a further bead incorporating embodiment of the invention;

FIG. 10a to 10c show different stages in the life of a device according to the invention, from initial point of deployment, through an intermediate time to a much later time after deployment;

FIG. 11 shows another embodiment of the invention in perspective view with the filling elements being inserted;

FIG. 12 shows stiffening elements incorporated according to another embodiment of the invention;

FIG. 13 shows an alternative form of stiffening; and

FIG. 14 shows a form of anchoring between the implant and surrounding tissue.

### DESCRIPTION OF SPECIFIC EMBODIMENTS

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure. The devices and methods disclosed herein boast a variety of inventive features and components that warrant patent protection, both individually and in combination.

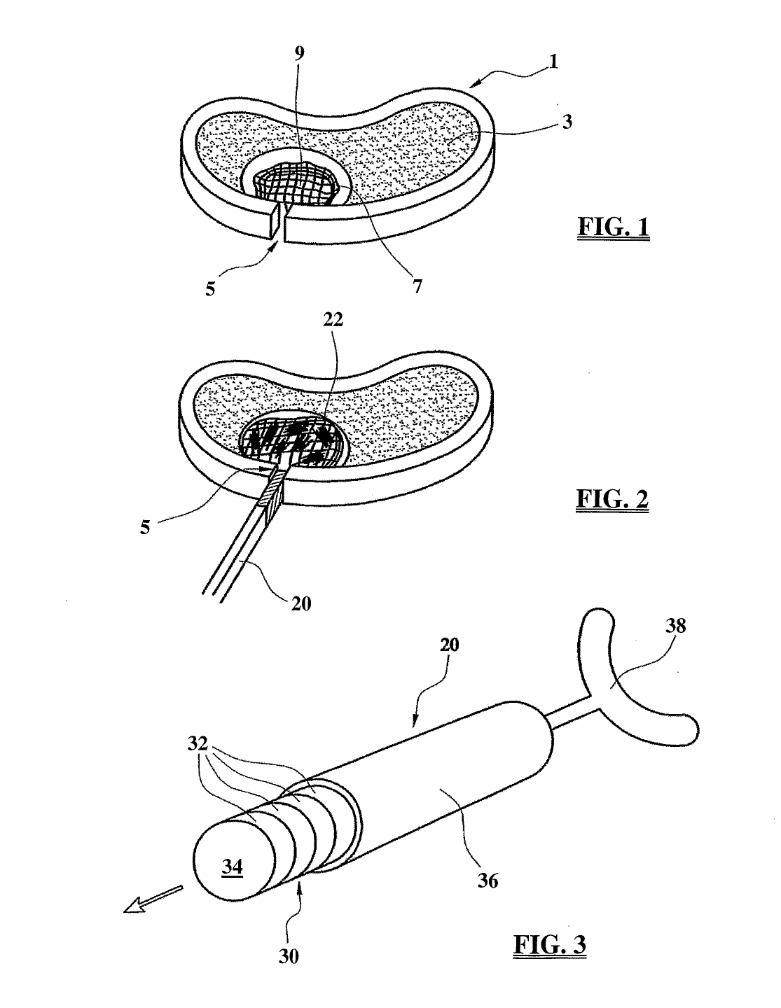

With reference to FIG. 1, an intervertebral disc 1 is shown with part of the nucleus 3 removed though an incision 5. Following removal of the material, the first part of the implant has been inserted. The first part is a fabric bag 7 with an opening 9. The bag 7 is empty and hence easily reduced to a small size at this stage so as to allow easy insertion through the incision 5. The incision 5 is of the smallest size necessary to remove the nucleus material. This comes with prior art systems where the incision 5 needed for the nucleus removal needed to be enlarged to allow enough room to deploy the implant. The opening 9 into the bag is kept close to the incision 5. The bag 7 is formed in such away as to offer the necessary strength and structural properties to constrain the filling it is to receive, but does so whilst being open to the passage of fluid through it, both into and out of its inside. The significance of this will be described in greater deal below.

In FIG. 2, the next stage of the implants formation is shown. Using an applicator 20, the second part of the implant, the filling 22 is pushed into the bag 7 though the opening 9. The filling 22 is of relatively small cross-section and so does not necessitate any enlargement of the incision 5 either. Sufficient filling 22 is introduced into the bag 7 to give it the desired properties discussed in more detail below. As can be seen, however, the filling 22 causes the bag 7 to generally assume the profile of the space in the nucleus 3. The filing 22 is made of one or more material which encourage tissue growth, such as polyester fibre, A bag such as bag 7 can be provided together with (as in used alongside but discrete from) or linked to or as an integral part of the type of device disclosed in applicant's PCT/GB200S/001 157 patent application, the contents of which are incorporated herein by reference with respect to that device. An implant according to the present invention is suitable when a procedure such as a nucleotomy has been conducted as the disc will have lost material from the nucleus. This may cause a loss in nucleus function and/or a loss in disc height. The implant thus provides a partial artificial and so provides treatment in these cases.

An important part of the present invention is the filling 22 used and structure of the bag 7. In disc/nucleus replacement procedures, the prior art approach has been to provide a non-biological mechanism for mimicking the disc's natural function throughout the life of the device. As far as practically possible the device has been isolated from its biological surrounds. The present invention aims to provide a phased transition from a solution based on a non-biological mechanism to a combination of biological and non-biological mechanisms and potentially even on to a predominantly or even exclusively biological mechanism. This aim can be achieved by careful design of the filling 22 and bag 7 to facilitate rather than resist tissue ingrowth. When exposed to alien materials which cannot be expelled or broken down, the bodies reaction is to try and isolate the material. Tissue thus grows around the material.

In the past, the continuous nature of the implant has meant that the tissue has grown only around the outside of the implant. In the case of inflatable balloons, this is because the outer which constrains the inflation, by its very nature, also prevents tissue growth inside. Similarly metal devices prevent tissue ingrowth because of the material they are made from. Other implants have used an outer which is continuous in nature and so only a surface layer of tissue around the very outside may have developed. Either because to the nature of the implant or because of active steps taken, no tissue ingrowth within the implant occurs, in some cases, steps to actively avoid tissue ingrowth have been taken, for instance to prevent the tissue interfering with the operation of the non-biological mechanics of the device. The present invention takes a fundamentally different approach and actively seeks tissue ingrowth for the implant.

Firstly, the bag 7 is provided in such a way that there are significant openings/gaps between the fibres forming the bags. Fluids can thus readily pass through the bag 7 in either direction. As a result the outer of the implant facilitates tissue ingrowth through itself. Secondly, and with reference to the FIG. 2 embodiment, the filling 22 consists of groups of fibres collected together in an unconstrained, unbraided mass. The elongate nature of the fibres suits them to alignment within the applicator 20. Some alignment is retained within the bag 7, but generally the result is a filling 22 formed of an open mass of fibres. Such a filling 22 of unconstrained and unbraided polyester filaments or fibres initially occupies a small volume in the nucleus. Following implantation, however, tissue ingrowth into the filling 22 occurs. The open nature of the mass of fibres and material of the fibres promotes this. With time, the tissue ingrowth tends to surround each fibre individually, as the tissue is able to reach each individually. Thus, each individual fibre is alien material to be isolated by surrounding. If densely packed fibres are provided, the tissue growth is again restricted to the outside as the fibres are seen as an integral mass by the tissue. The open fibres of the present invention in effect act as a scaffold. As this growth progresses, it will cause the volume of the filling 22 and hence the bag 7 to swell to fill the available space in the nucleus.

The lack of restriction on the tissue ingrowth and the free access for fluids into and out of the bag 7 and filling 22 should mean that the tissue which grows is similar in composition and hence properties to the undisturbed nucleus material that surrounds it. The swelling of the bag 7 should restore some of the disc height that has been lost as the disc failed. In theory, during the earlier stages of degenerative disc disease, the idea of refilling the nucleus with scaffolding polyester fibre could act as a permanent treatment. At the very least, it would be expected to improve the patient's condition in the medium term delaying a more serious procedure. In the meantime, all normal treatment options would still be able to be used on the patient.

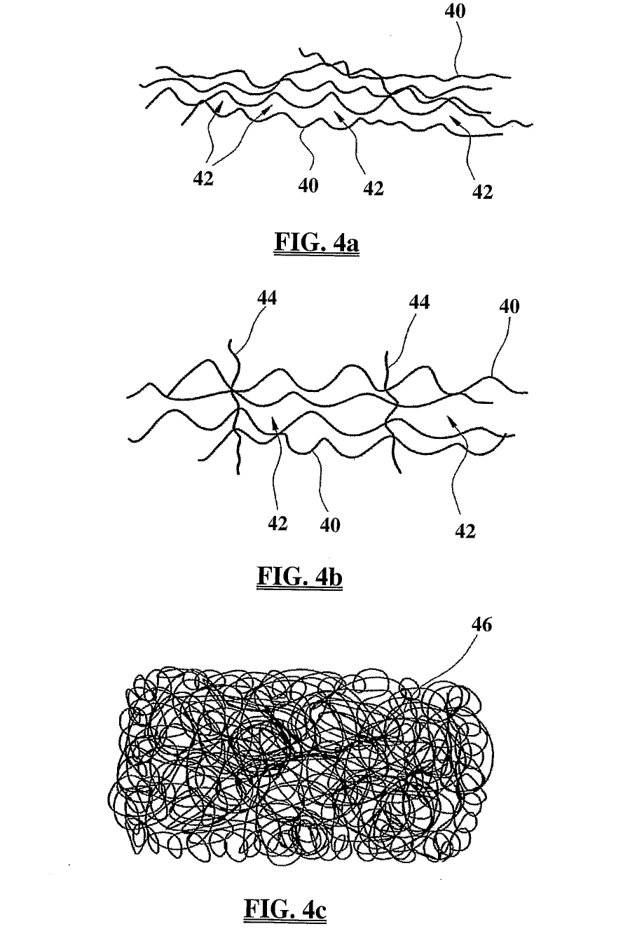

The applicator 20 is illustrated in more detail in FIG. 3, in conjunction with a different form of filling 30, to the filling 22 used in the FIG. 2 embodiment. In this case, rather than being a mass of fibres in an unconstrained form, the filling 30 is provided in the form of a number of discrete pads 32 of felt like material 34. Felt and similar materials used the natural interlacing of their fibres to form an open porous structure. This can be supplemented by needling to increase the interlacing and/or openness of the structure if desired. The applicator 20 consists of a tube 36 which holds the pads 32. Under the control of the surgeon a plunger 38 is advanced in the tube 36 to push the pads 32 out into the bag 7 within the disc. Overtime, the pads 32 expand as tissue grows within and around them. Different applicator cross-sections can be used to deploy different fillings.

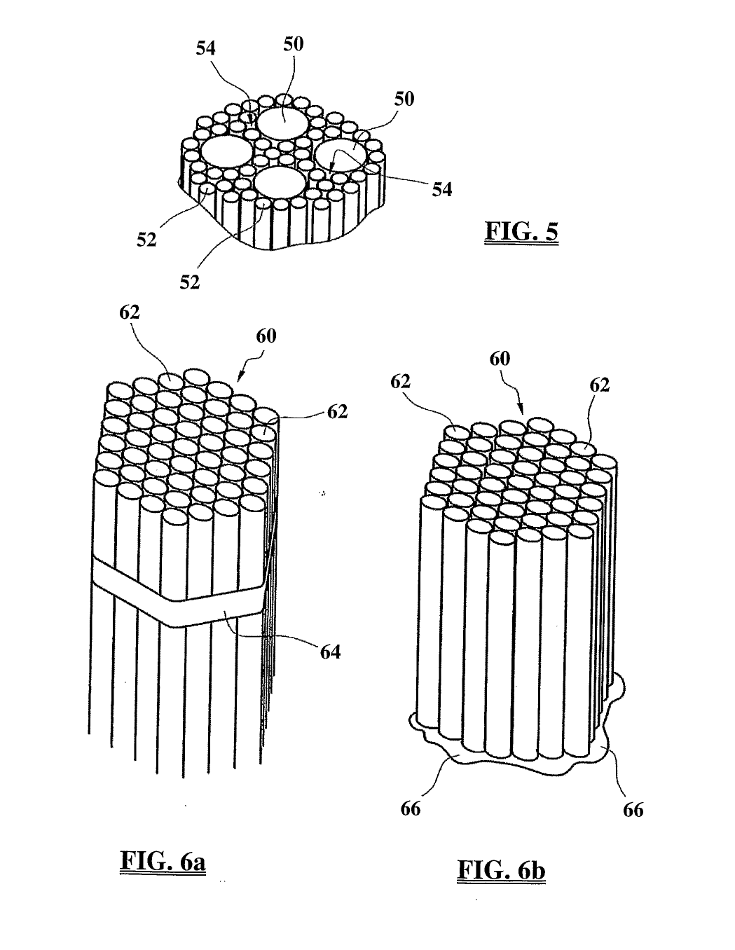

FIGS. 4a, 4b and 4c illustrate a number of alternative forums of filling 22 in unconstrained, unbraided form. FIG. 4c shows a series of generally aligned fibres 40 which are non-linear in nature. The waves built into the fibres 40 serve to space individual fibres 40 from one another. The result is a mass of fibres 40 with substantial voids 42. FIG. 4b shows a modification, in which a series of secondary fibres 44 are provided with a different orientation to the primary fibres 40. The difference in orientation resists pressures which would otherwise cause the voids 42 between the fibres to be reduced. FIG. 4c shows a mass of fibres 46 in a form more closely approaching that of a felt or cotton wool material. A very large number of different orientations are provided and thus serve to maintain the spacing against compression in a wide variety of directions. The fibre could be provided from staple fibre, potentially subsequently chopped into short lengths. The fibre could be used as supplied or be modified before or after chopping, potentially to provide braiding or other restraining surround. It is possible to use fibres form of single filaments and/or filaments twisted together and/or braided together.

FIG. 5 shows a further filling form in which primary fibres 50 of a large cross-section are mixed with secondary fibres 52 of a smaller cross-section. The differences in cross-section again help to maintain the voids 54 within the filling.

FIGS. 6a and 6b illustrate examples of a more structured filling 60. In the first case, FIG. 6a, the majority of the fibres 62 are provided along a first alignment. To assist in keeping the alignment of the fibres 62 during and after deployment, a limited number of fibres 64 are wrapped around the fibres 62 to maintain them as bundles. The bundles are still open, however, and have significant voids. In the FIG. 6b form, the fibre bundle is chopped by a hot blade and this melts part of the ends and joins them together upon cooling due to mass 66.

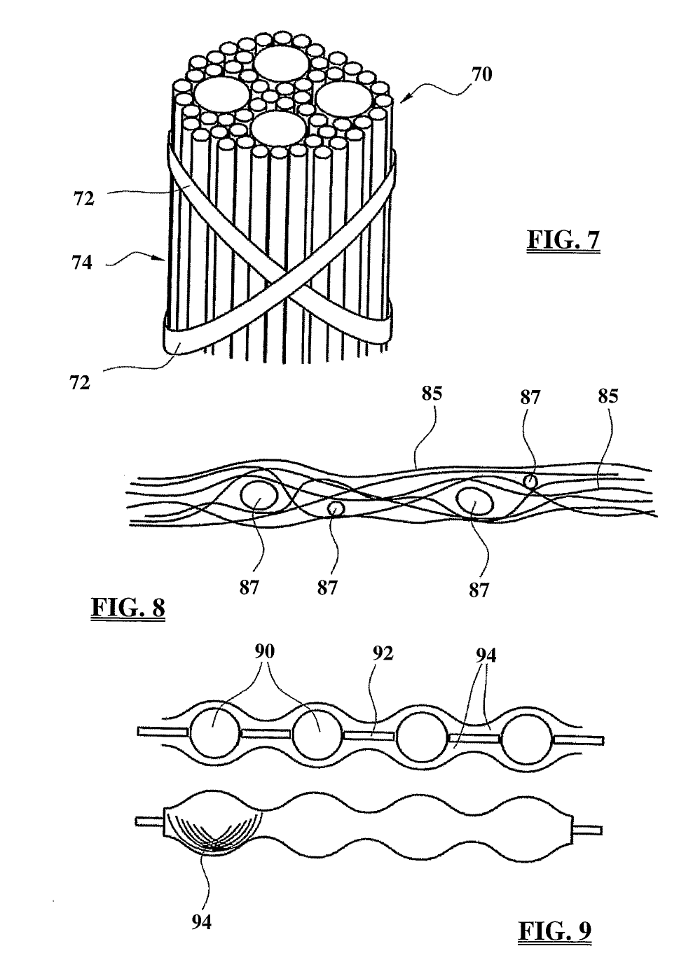

The FIG. 7 embodiment is a still more structured embodiment of the filling 70. An outer layer of criss-cross fibres 72 is provided so as to maintain the inner fibres 74 in the desired position. The inner fibres 74 are a mixture of large 76 and small 78 fibres, By potentially providing the fibres 76, 78 on a number of slightly different alignments a more open structure with large voids is provided. The large gaps in the outer layer of crisscross fibres 72 means that there is no interference with tissue ingrowth, but these fibres can be provided with a degree of stiffness to assist deployment and positioning of the filling 70 within the space in the bag which surrounds it. A series of lengths of such filling 70 can be used in a single bag to give the desired overall structure.

In FIG. 8, the fibres 85 within a bundle are spaced and provided on a variety of alignments by the inclusion of a number of spherical beads 87. Finally in FIG. 9, a series of beads 90 are provided linked together by a fibre 92. The heads are each surrounded by a mass of fibres 94 braided on to form a mass. The braided mass 94 surrounds each of the beads 90 like a sleeve. Again the filling itself is open and promotes tissue growth.

In many of the above cases, the desired open structure is not only provided by individual groups of fibres, but also by the interaction between individual groups of fibres and the voids between them that they define.

In all of the above embodiments, and in the invention in general, the provision of an open structure can also be assisted by the careful use of different materials for different parts of the filling.

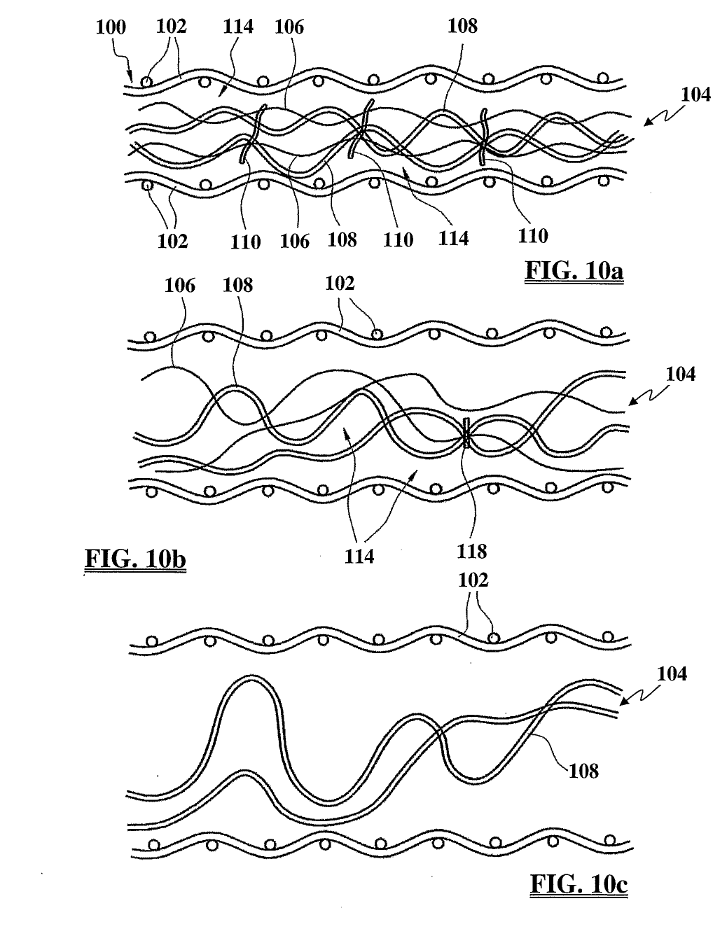

At the time of deployment, FIG. 10a, the bag 100 is formed of a number of fibres 102 woven together to provided the necessary structure for containing the filling 104. The filling 104 itself is provided in the form of a series of wavy fibres of a first size 106 and second size 108, together with spacing fibres 110 which assist in maintaining the open position of the first size 106 and second size 108 fibres under compression. The result is an open structure with substantial gaps in the bag 100 to allow fluid communication through the bag 100 and substantial voids 114 between the fibres 106, 108, 110.

Six months or so after deployment, FIG. 10b, the position has changed. Substantial amounts of tissue ingrowth has occurred. The tissue ingrowth serves in effect to provide nucleus material which resists compression of the nucleus and filling 104. The spacing fibres 110 are no longer required, therefore, having served their function of resisting compression of the fibres 106, 108 during the early days of the implant. By providing the spacing fibres 110 from a bio-absorbable material which is relatively quickly absorbed, within 6 months or so, the spacing fibres 110 are removed from the equation. The tissue they served as a scaffold for usefully remains, but the fibres 110 themselves have gone in most places. A few remains 118 of such fibres 110 may remain. As a result of these fibres 110 going, there is no restriction on the expansion of the voids 114 by the spacing between fibres 106, 108 increasing. The tissue growth itself provides the expansive pressure for this to happen. The non-bioabsorbable fibres 102 of the bag 100 remain, as do the fibres 106, 108 to provide assistance to the overall structure.

FIG. 10c shows the position some 2 years or so after deployment. Yet further tissue growth has occurred and the regenerated tissue now provides the majority of the nucleus function. With this mainly biological provision of the necessary structure, there is less need for the fibres 106, 108. As the fibres 106 are also provided from a bioabsorbable material, these too are disappearing. Different time periods for bio-absorption to occurred are possible though selection of the material used. The removal of the fibres 106 allows the remaining fibres 108 to expand still further.

So as to accurately gauge the size of bag required and amount of filling needed, it is possible to measure the inflated volume of an inflatable bag inserted into the space vacated by the removed nucleus material.

As described in the previous embodiments, the implant is generally in the form of a bag and filling. However, in certain case it may be possible to use the filling without a bag. This is particularly the case where the removal of the disc material to form the void which needs filling is well defined, for instance due to its being bounded by the natural annulus and/or sound nucleus material.

In such cases, the benefits according to the present invention are still provided due to the different approach to the replacement of the nucleus material taken through its replacement by fibres. Again the present invention aims to provide a phased transition from a solution based on a non-biological mechanism to a combination of biological and non-biological mechanisms and potentially even on to a predominantly or even exclusively biological mechanism.

Fibres of the types, materials and configurations described above can be used in this embodiment. Once again, when exposed to such alien materials the body's reaction is to try and isolate the material by providing tissue growth around it.

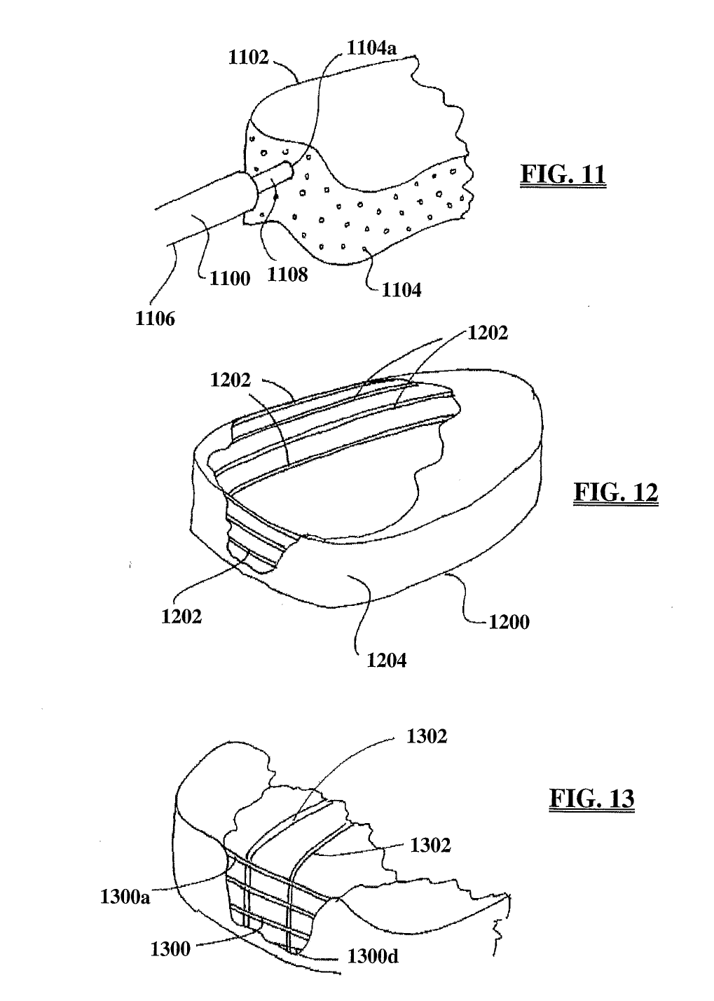

FIG. 11 illustrates a further embodiment of the invention with the filling 1100 being introduced into the outer component 1102. In this instance, the outer component 1102 is not provided with an opening in an end (see FIG. 1), but instead is filled though one of the pores 1104 in the outer component 1102. The filling element 1100, in this case a flowable material, is provided in an injection tool 1106. The tip 1108 of the injection tool 1106 is of reduced diameter and so can be pushed into a pore 1104a. In doing so the size of the pore I 104a is increased by stretching to be greater than the size of the tip 1108. The filling 1100 can then be injected. The viscosity of the filling 1100 is such that it can readily flow under pressure from the injection tool 1106 through an opening of size 1104a. However, the viscosity is such that it cannot readily flow through an opening of size 1104, particularly when under the lower pressure levels experienced within the outer component 1102 compared with those experienced in the tip 1108. The same principle would apply where the filling includes distinct particles such as beads or fibres. The stretched pore is large enough to allow the filling in, but the normal size pores are too small to allow the filling out.

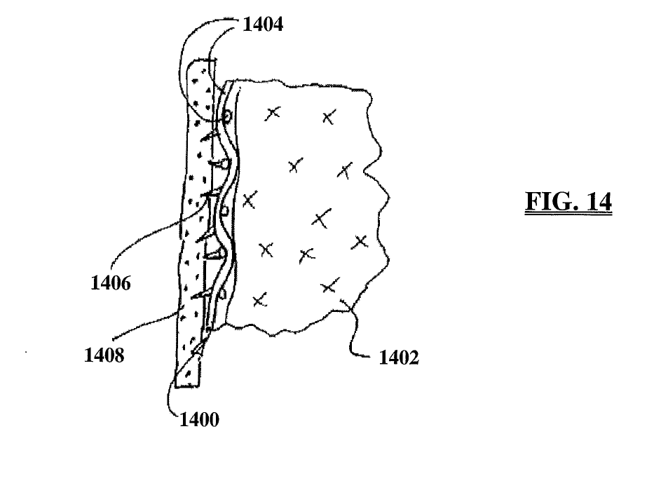

While the outer component can be entirely flexible, consistent with its fabric/textile nature, there are benefits it providing a more defined structure or profile to the outer component. Thus in the embodiment of FIG. 12 the outer component 1200 is provided of fabric, but within the bag a number of stiffening elements 1202 are provided. Thus a series of stiffening elements 1202a are provided in the form of rings which extend around the periphery of the outer component 1200 and so seek to maintain the side wall 1204 profile of the outer component 1200. For insertion, the sides of the rings can be squeezed together and so reduce the cross-section of the outer component 1200. Once inside the disc space, the compression can be removed and the rings will push the sides 1204 of the outer component 1200 outwards to the disc like profile. This assists in ensuring the shape of the implant is correct and assists in providing the space into which the filling elements can be introduced.

In the FIG. 13 detail, the stiffening elements 1300 are supplemented by stiffening elements 1302 which seek to push the top 1300a and bottom 1300d rings apart in a vertical sense. Again a downward compression can be used to reduce the profile of the outer component 1300, with the removal of that compression allowing the outer component 1300 to return to the desired form. Such arrangements of stiffening elements can be used to close or assist in supporting the closure of an outer component. Equally such stiffening elements can be used to support surfaces of the outer component against loads. For instance, the surface of the implant which faces the vertebra above the implant in a standing individual and/or the surface which faces the vertebra below may be provided with stiffening elements which extend across them to resist loads. Stiffening elements down the sides, round the edges and at other positions may also be provided to support the shape of the implant and/or contribute to its functional characteristics. Resistance to load, extension, compression, flexion or the like may be provided in this way, as might resistance to tissue ingrowth pressures.

The characteristics provided by the stiffening elements may be different for different parts of the implant. For instance, some parts may be less resistant to a force than others. Metal wires, metal fibres, stiff plastics wires or fibres and the like could be used for the stiffening elements. In particular shape memory materials, such as nitinol, could be used for the stiffening elements. A wide variety of configurations are possible, including rings, spirals, zig-zags, loops, coils, waves and others.

In the embodiment illustrated in FIG. 14, the detailed view shows a cross-section though the outer component 1400 and filling 1402 which in this case is a hydrogel. The fibres 1404 which are woven together to form the outer component 1400 include on their outside a series of projections 1406. These projections 1406 are integrally formed with the fibres 1404 and are provided at an inclined angle. As such as the outer component 1400 expands during filling and/or moves during insertion/filling/use, the projections 1406 act as barbs and dig into the surrounding material of the annulus 1408. In this way a firm anchorage for the implant is provided all over its surface, including those parts which could not be reached from the small incision used to insert the implant. If sutures or staples are to be used to fix the implant within the annulus, then they can only really be provided at or close to the incision site.

While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed, but on the contrary, the invention is to cover all modifications, equivalents, and alternatives falling within the spirit and scope of the invention as defined herein.

1. A disc prosthesis comprising:
an outer component manufactured at least in part from fabric; and
at least one filling element being introduced into said outer component.

2. The disc prosthesis of claim 1, wherein at least one of fibrous material and human tissue.

3. The disc prosthesis of claim 1, wherein at least one filling element is at least one of porous, defines voids between at least one other filling element, and defines voids between parts of a filling element.

4. The disc prosthesis of claim 1, wherein at least one filling element is formed of at least one of unconstrained fibers, unbraided fibers, interlaced fibers, wavy fibers, curved fibers, zig-zag fibers, and fibers having different cross-sections.

5. The disc prosthesis of claim 1, wherein at least one of the top surface and bottom surface of the outer component is at least one of octagonal, hexagonal, round, and elliptic.

6. The disc prosthesis of claim 1, wherein the outer component includes at least one stiffening element.

7. The disc prosthesis of claim 6, wherein said at least one stiffening element is disposed at least partially around the periphery of said outer component.

8. The disc prosthesis of claim 1, wherein at least one of the outer component and the at least one filling element is formed with at least one material intended to promote tissue growth.

9. The disc prosthesis of claim 1, wherein at least one of said outer component and said at least one filling element is formed with at least one bio-absorbable material.

10. The disc prosthesis of claim 1, wherein the outer component includes a top wall, a bottom wall, and a plurality of side walls connected to at least one of the top wall and bottom wall.

11. The disc prosthesis of claim 1, wherein said at least one filling element is introduced into said outer component through at least one of pre-existing pores in said outer component created during the manufacturing process, apertures created in said outer component during the introduction of said at least one filling element into said outer component, and at least one flap that may be selectively manipulated to open an interior region of said outer component to introduce said at least one filling element into said outer component.

12. The disc prosthesis of claim 11, wherein said pores of said outer component are dimensioned to prevent said at least one filling element from migrating out of said outer component after said at least one filling element has been introduced into said outer component.

13. The disc prosthesis of claim 1, wherein at least one of said outer component and said at least one filling element is equipped with loops.

14. The disc prosthesis of claim 10, wherein at least one of the top wall and bottom wall of the outer component includes at least one edge provided with at least one flange, the at least one flange providing at least one anchor location for attaching the outer component to at least one vertebrae.

15. The disc prosthesis of claim 6, wherein said at least one stiffening element is comprised of at least one of metal wire, metal fiber, stiff plastic wire, shape memory material and shaped in at least one of a ring, spiral, zig-zag, loop, coil, and wave.

16. The disc prosthesis of claim 1, wherein said outer component includes a plurality of projections to extend into adjacent tissue when the outer component is implanted.

17. A kit for use in spine surgery, comprising:
a series of different sized disc prostheses within one or more containers, wherein each disc prosthesis includes an outer component manufactured at least in part from fabric and at least one filling element being introduced into said outer component.

18. The kit of claim 17, wherein the at least one filling element is at least one of fibrous material and human tissue.

19. A method for performing spine surgery, comprising the steps of:
(a) removing at least part of the natural disc in a spine to create a space; and
(b) inserting a disc prosthesis into said space, said disc prosthesis including an outer component manufactured at least in part of fabric and at least one filling element being introduced into said outer component.

20. A method of claim 19 wherein the at least one filling element is at least one of fibrous material and human tissue.

21. The kit of claim 17, wherein at least one filling element is at least one of porous, defines voids between at least one other filling element, and defines voids between parts of a filling element.

22. The kit of claim 17, wherein at least one filling element is formed of at least one of unconstrained fibers, unbraided fibers, interlaced fibers, wavy fibers, curved fibers, zigzag fibers, and fibers having different cross-sections.

23. The kit of claim 17, wherein at least one of the top surface and bottom surface of the outer component is at least one of octagonal, hexagonal, round, and elliptic.

24. The kit of claim 17, wherein the outer component includes at least one stiffening element.

25. The kit of claim 24, wherein said at least one stiffening element is disposed at least partially around the periphery of said outer component.

26. The kit of claim 24, wherein said at least one stiffening element is comprised of at least one of metal wire, metal fiber, stiff plastic wire, shape memory material and shaped in at least one of a ring, spiral, zigzag, loop, coil, and wave.

27. The kit of claim 17, wherein at least one of the outer component and the at least one filling element is formed with at least one material intended to promote tissue growth.

28. The kit of claim 17, wherein at least one of said outer component and said at least one filling element is formed with at least one bio-absorbable material.

29. The kit of claim 17, wherein the outer component includes a top wall, a bottom wall, and a plurality of side walls connected to at least one of the top wall and bottom wall.

30. The kit of claim 29, wherein at least one of the top wall and bottom wall of the outer component includes at least one edge provided with at least one flange, the at least one flange providing at least one anchor location for attaching the outer component to at least one vertebrae.

31. The kit of claim 17, wherein said at least one filling element is introduced into said outer component through at least one of pre-existing pores in said outer component created during the manufacturing process, apertures created in said outer component during the introduction of said at least one filling element into said outer component, and at least one flap that may be selectively manipulated to open an interior region of said outer component to introduce said at least one filling element into said outer component.

32. The kit of claim 31, wherein said pores of said outer component are dimensioned to prevent said at least one filling element from migrating out of said outer component after said at least one filling element has been introduced into said outer component.

33. The kit of claim 17, wherein at least one of said outer component and said at least one filling element is equipped with loops.

34. The kit of claim 17, wherein said outer component includes a plurality of projections to extend into adjacent tissue when the outer component is implanted.

35. The method of claim 19, wherein at least one filling element is at least one of porous, defines voids between at least one other filling element, and defines voids between parts of a filling element.

36. The method of claim 19, wherein at least one filling element is formed of at least one of unconstrained fibers, unbraided fibers, interlaced fibers, wavy fibers, curved fibers, zigzag fibers, and fibers having different cross-sections.

37. The method of claim 19, wherein at least one of the top surface and bottom surface of the outer component is at least one of octagonal, hexagonal, round, and elliptic.

38. The method of claim 19, wherein the outer component includes at least one stiffening element.

39. The method of claim 38, wherein said at least one stiffening element is disposed at least partially around the periphery of said outer component.

40. The method of claim 38, wherein said at least one stiffening element is comprised of at least one of metal wire, metal fiber, stiff plastic wire, shape memory material and shaped in at least one of a ring, spiral, zigzag, loop, coil, and wave.

41. The method of claim 19, wherein at least one of the outer component and the at least one filling element is formed with at least one material intended to promote tissue growth.

42. The method of claim 19, wherein at least one of said outer component and said at least one filling element is formed with at least one bio-absorbable material.

43. The method of claim 19, wherein the outer component includes a top wall, a bottom wall, and a plurality of side walls connected to at least one of the top wall and bottom wall.

44. The method of claim 43, wherein at least one of the top wall and bottom wall of the outer component includes at least one edge provided with at least one flange, the at least one flange providing at least one anchor location for attaching the outer component to at least one vertebrae.

45. The method of claim 19, wherein said at least one filling element is introduced into said outer component through at least one of pre-existing pores in said outer component created during the manufacturing process, apertures created in said outer component during the introduction of said at least one filling element into said outer component, and at least one flap that may be selectively manipulated to open an interior region of said outer component to introduce said at least one filling element into said outer component.

46. The method of claim 45, wherein said pores of said outer component are dimensioned to prevent said at least one filling element from migrating out of said outer component after said at least one filling element has been introduced into said outer component.

47. The method of claim 19, wherein at least one of said outer component and said at least one filling element is equipped with loops.

48. The method of claim 19, wherein said outer component includes a plurality of projections to extend into adjacent tissue when the outer component is implanted.
